(19) United States
(12) Patent Application Publication
(10) Pub. No.: US 2016/0247301 A1
(43) Pub. Date: Aug. 25, 2016

(54) LIGHT DETECTION APPARATUS AND IMAGE RECONSTRUCTION METHOD USING THE SAME

(71) Applicant: National Chiao Tung University, Hsinchu (TW)

(72) Inventors: Wai-Chi Fang, Hsinchu (TW); Hsiang-Wen Hou, Hsinchu (TW); Hao-Jan Sun, Hsinchu (TW)

(21) Appl. No.: 14/829,144

(22) Filed: Aug. 18, 2015

(30) Foreign Application Priority Data
Feb. 25, 2015 (TW) 104106003

(51) Int. Cl. G06T 11/00; H04N 5/33; A61B 5/00; G06T 7/00

(52) U.S. Cl. CPC G06T 11/006; G06T 7/0012; H04N 5/33; A61B 5/0073; G06T 2211/428; G06T 2207/30004; G06T 2207/10101

(57) ABSTRACT

A light detection apparatus and an image reconstruction method using the light detection apparatus are provided. The light detection apparatus includes a detection module and a control module. The detection module has a plurality of light detection units to constitute a hexagonal or honeycomb array structure. Each of the light detection units has a light-emitting element and a photosensitive element. The control module has a selector and a multiplexer. The selector selects at least one light-emitting element to produce a light source, so as to emit a plurality of photons to an object-under-test. The multiplexer selects at least one photosensitive element to detect light signals of the photons diffused to the object-under-test. The invention can obtain more light signals from the object-under-test to reconstruct images of the object-under-test.

### BACKGROUND OF THE INVENTION

1. Field of the Invention

The present invention relates to light detection apparatuses and image reconstruction methods, and, more particularly, to a light detection apparatus with a hexagonal or honeycomb array structure and an image reconstruction method using the light detection apparatus.

2. Description of Related Art

Diffuse Optical Tomography (DOT) is a new non-invasive technique that has been widely used in clinical diagnosis. Functional Near-Infrared Ray (FNIR) is one of the important techniques in DOT and has been used in two-dimensional image reconstruction because of its good time and spatial resolutions.

Furthermore, home healthcare products demand portability, low cost and immediate image realization. However, current image reconstruction techniques rely rather heavily on computer and software interfaces and require large amounts of matrix operations in order to achieve high resolution. A great number of operations result in long image reconstruction time, not meeting the need for real-time and fast reconstruction, and hinder the application of home health care system.

In addition, conventional light detection apparatus usually employs quadrilateral array structure, such that one light emitting element of the light detection apparatus only corresponds to photosensitive elements in a maximum of four different directions, so that the light-detecting apparatus extracts fewer light signals from an object-under-test and is unfavorable to the reconstruction of the image of the object-under-test.

Therefore, there is a need for a solution that address the aforementioned shortcomings in the prior art.

### SUMMARY OF THE INVENTION

The present invention provides a light detection apparatus and an image reconstruction method using the same, which allow more light signals to be retrieved from an object-under-test in order to reconstruct an image of the object-under-test.

The light detection apparatus of the present invention may include a detection module including a plurality of light detection units forming a hexagonal or honeycomb array structure, each of the light detection units including at least one light-emitting element and a photosensitive element; and a control module connected with the detection module and including at least one selector and a multiplexer, wherein the selector selects at least one of the light-emitting elements of the light detection units to allow the selected light-emitting element to produce a light source and emit a plurality of photons to an object-under-test, and the multiplexer selects at least one of the photosensitive elements of the light detection units to allow the selected photosensitive element to detect light signals of the photons diffused to the object-under-test.

In an embodiment, each of the light detection units has a hexagonal grid or border, and each light-emitting element of the light detection units is adjacent to six photosensitive elements at most. The light-emitting elements or the photosensitive elements in the same row of the light detection units are closely spaced at intervals of multiple increments.

In another embodiment, each light-emitting element of the light detection unit includes two light-emitting diodes (LEDs) that provide two light sources with two wavelengths, and the control module includes two selectors, which control the two light sources of the light-emitting element of the light detection unit. The multiplexer is connected with the photosensitive elements of the light detection units for receiving light signals detected by these photosensitive elements.

In yet another embodiment, the light detection apparatus may include a conversion module connected with the multiplexer for converting light signals from light intensity signals to voltage signals. The light detection apparatus may also include a processing module connected with the conversion module for constructing an image of a tissue structure of the object-under-test based on the voltage signals converted by the conversion module.

Moreover, the image reconstruction method using the light detection apparatus may include: allowing the light detection units of the light detection apparatus to correspond to the object-under-test; setting a plurality of first initial values based on the light detection units and the relative location of a first-layer tissue structure at a first depth of the object-under-test; and using a first iteration algorithm to calculate a plurality of first image values for the first-layer tissue structure based on the first initial values, first optical paths between the light-emitting elements and adjacent photosensitive elements, and the light signals detected by these adjacent photosensitive elements, to amend the first images values repeatedly until the first image values are smaller than a first threshold, and constructing a first image based on the first image values.

In an embodiment, the image reconstruction method may include: setting a plurality of second initial values based on the light detection units and the relative location of a second-layer tissue structure at a second depth of the object-under-test; and using a second iteration algorithm to calculate a plurality of second image values for the second-layer tissue structure based on the first image values, the second initial values, second optical paths between the light-emitting elements and photosensitive elements that are spaced apart at two intervals, and the light signals detected by the two-interval spaced photosensitive elements, to amended the second images values repeatedly until the second image values are smaller than a second threshold, and constructing a second image based on the second image values.

In another embodiment, the image reconstruction method may include: setting a plurality of third initial values based on the light detection units and the relative location of a third-layer tissue structure at a third depth of the object-under-test; and using a third iteration algorithm to calculate a plurality of third image values for the third-layer tissue structure based on the third image values, the third initial values, third optical paths between the light-emitting elements and photosensitive elements that are spaced apart at three intervals, and the light signals detected by the three-interval spaced photosensitive elements, to amend the third images values repeatedly until the third image values are smaller than a third threshold, and constructing a third image based on the third image values.

From the above, it is known that the light detection units of the detection module are constructed in such a way that they form a hexagonal or honeycomb array structure, so that the light source of each light-emitting element corresponds to photosensitive elements in six different directions simultaneously based on the characteristic of closely stacked hexagons. Therefore, the light detection apparatus is able to detect more light signals from the object-under-test, thus enabling fast reconstruction of the image of the object-under-test, and at the same time allowing the image of the object-under-test to have high resolution. Meanwhile, the light detection apparatus is portable and low cost, and is capable of Multiple-Input Multiple Output (MIMO) through the plurality of light-emitting elements and the plurality of photosensitive elements.

Furthermore, in the image reconstruction method using the light detection apparatus according to the present invention, in addition to capable of detecting more light signals from the object-under-test, a first image of a first-layer tissue structure to a third image of a third-layer tissue structure of the object-under-test can be respectively constructed based on the first to the third iteration algorithms, thus facilitating the reconstruction of an image (e.g., a 3D image) of the object-under-test that is three layers deep.

In addition, the light detection apparatus of the present invention and the image reconstruction method using the same can be applied to diffuse optical tomography (DOT) systems, remote real-time monitoring care systems (such as home healthcare systems), relevant medical systems or other areas in order to provide the detections of breast cancer lesions or hemorrhagic stroke or the verification of brain functions, allowing users (such as physicians) to determine if the tissue structures of the object-under-test are normal or not based on these images and to quickly grasp a patient's condition or have real-time information concerning the situation of an individual being looked after.

### BRIEF DESCRIPTION OF DRAWINGS

FIG. 1 is a block diagram illustrating a light detection apparatus in accordance with the present invention;

FIG. 2 is a schematic diagram illustrating a detection module of the light detection apparatus of FIG. 1 in accordance with the present invention;

FIG. 3 is a flowchart illustrating an image reconstruction method using the light detection apparatus of FIGS. 1 and 2 in accordance with the present invention;

FIG. 4 is a schematic diagram depicting the detection module of FIG. 2 corresponding to the object-under-test and a first optical path to a third optical path in accordance with the present invention;

FIG. 5 is a schematic diagram depicting the detection module of FIG. 2 corresponding to the object-under-test and a plurality of first initial values to third initial values in accordance with the present invention; and

FIGS. 6A to 6C are schematic diagrams depicting a first image for a first-layer tissue structure, a second image for a second-layer tissue structure, and a third image for a third-layer tissue structure, respectively, of the object-under-test in accordance with the present invention.

### DETAILED DESCRIPTION OF THE INVENTION

The present invention is described by the following specific embodiments. Those with ordinary skills in the arts can readily understand other advantages and functions of the present invention after reading the disclosure of this specification.

It should be noted that the structures, proportions, sizes and the like shown in the attached drawings are to be considered only in conjunction with the contents of this specification and to facilitate understanding and reading by those skilled in the art. They are not intended to limit the scope of present invention, thus holds no technically significance. Any changes or modifications in the structures, the proportions, the sizes and the like should fall within the scope of the technical contents disclosed in the present invention as long as they do not affect the effects and the objectives achieved by the present invention.

Meanwhile, terms such as “first”, “second” and “connection” used in this specification are used for illustration purposes only, and are not intended to limit the scope of the present invention in any way, any changes or modifications of the relative relationships of elements are therefore to be construed as within the scope of the present invention as long as there is no substantial changes to the technical contents. Moreover, the term “connection” can be used to represent coupling, electrically connection, signal connection, wired connection, wireless connection, direct connection, indirect connection or so forth.

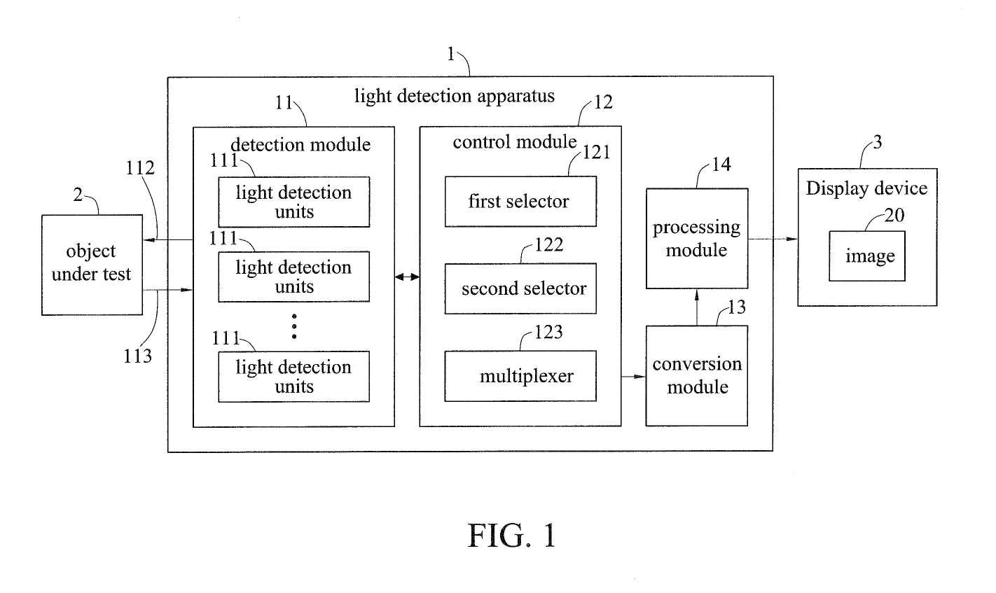

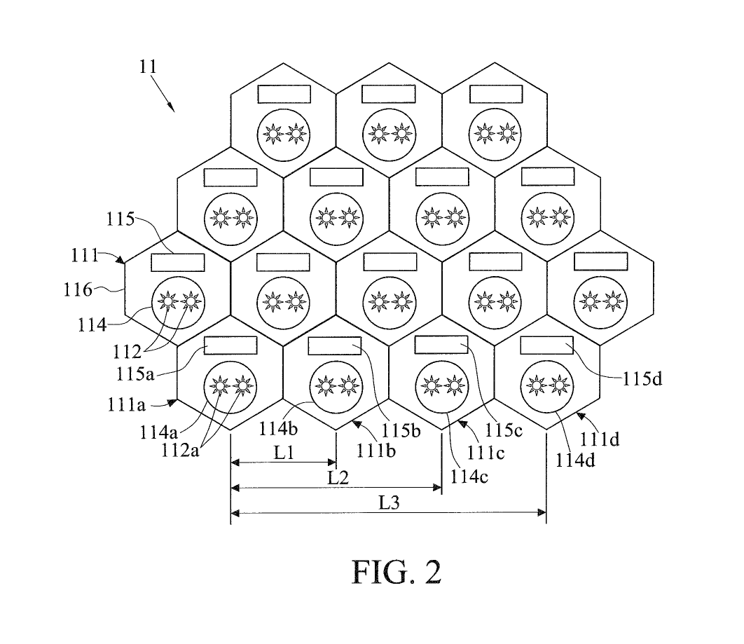

FIG. 1 is a block diagram illustrating a light detection apparatus 1 in accordance with the present invention. FIG. 2 is a schematic diagram illustrating a detection module 11 of the light detection apparatus 1 of FIG. 1 in accordance with the present invention.

As shown in FIGS. 1 and 2, the light detection apparatus 1 includes a detection module 11 and a control module 12. The light detection apparatus 1 may further include a conversion module 13 and a processing module 14.

The detection module 11 has a plurality of light detection units 111, forming a hexagonal or honeycomb array structure. Each of the light detection units 111 includes at least one light-emitting element 114 and a photosensitive element 115. The light-emitting element 114 may include, for example, a LED, and is capable of emitting Functional Near-Infrared Ray (FNIR) or other types of light. The photosensitive element 115 may be an optical sensor, a light diode or the like. In an embodiment, the detection module 11 includes 16 light detection units 111, 16 light-emitting elements 114, and 16 photosensitive elements 115. However, the number of light detection units 111, light-emitting element 114 or photosensitive element 115 can also be 32, 64 or more.

Each of the light detection units 111 may include a hexagonal grid 116 or border (sideline). One of the light-emitting elements 114 of a light detection unit 111 may be surrounded by six adjacent photosensitive elements 115 at most, wherein an “adjacent” element may mean the closest element or an element that is one interval L1 (e.g. 0.667 cm) away. Furthermore, there can be an equal interval L1 between the light-emitting elements 114, between the photosensitive elements 115, or between the light-emitting elements 114 and the photosensitive elements 115.

As shown in FIG. 2, the light-emitting elements 114 or the photosensitive elements 115 in the same row of the light detection units 111 can be closely spaced at intervals of incremental multiples. For example, a light-emitting element 114a is spaced from a light-emitting element 114b, a light-emitting element 114c, and light-emitting element 114d by one interval L1 (e.g., 0.667 cm), two intervals L2 (e.g. 1.334 cm) and three intervals L3 (e.g., 2 cm), respectively. Similarly, a photosensitive element 115a is spaced from a photosensitive element 115b, a photosensitive element 115c, and a photosensitive element 115d by one interval L1 (e.g., 0.667 cm), two intervals L2 (e.g., 1.334 cm) and three intervals L3 (e.g., 2 cm), but the present invention is not limited thereto.

The control module 12 is connected to the detection module 11, and includes at least one selector (e.g., 121 or 122) and a multiplexer 123. The selector is used for selecting at least one of the light-emitting elements 114 of the light detection units 111, so that the selected light-emitting element 114 produces a light source 112 and emits a plurality of photons (not shown) to an object-under-test 2. Then, the multiplexer 123 selects at least one of the photosensitive elements 115 of the light detection units 111 in order to detect light signals 113 of the photons diffused into the object-under-test 2 with the selected photosensitive element 115. The object-under-test 2 may be a human body, an animal body or other objects.

In an embodiment, each of the light-emitting elements 114 of the light detection units 111 may include two LEDs to emit two light sources 112 of two or different wavelengths. The two wavelengths may be 750 nm and 850 nm, for example. The selector includes a first selector 121 and a second selector 122. The first selector 121 may control one of the two light sources 112 of a light-emitting element 114 of a light detection unit. The second selector 122 may control the other one of the two light sources 112.

The first selector 121 or the second selector 122 may be a multiplexer (e.g., an analog multiplexer), a control chip (IC) and etc. The multiplexer 123 may be a demultiplexer (e.g., a digital demultiplexer) or a control chip. For example, the first selector 121, the second selector 122 or the multiplexer 123 may be binary 4-bit, 5-bit, 6-or-more-bit control chip that provides 16(2⁴), 32(2⁵), 64(2⁶) or more control signals to control 16, 32, 64 or more light-emitting elements 114 or photosensitive elements 115.

In addition, the multiplexer 123 may also be connected to the photosensitive elements 115 of the light detection units 111 to receive the light signals 113 detected by the photosensitive elements 115.

The conversion module 13 may be connected to the multiplexer 123 of the control module 12 for converting the light signals 113 (light intensity signals) received by the multiplexer 123 into voltage signals. The conversion module 13 may be an Analog-to-Digital Converter (ADC) or an analog-to-digital program or software.

The processing module 14 may be connected to the conversion module 13 for constructing an image 20 of the object-under-test 2 based on the voltage signals converted by the conversion module 13. The processing module 14 may transmit the image 20 of the object-under-test 2 to a display device 3 to be displayed. The processing module 14 may be a processor (hardware) or a processing program (software). The image 20 may be a three-dimensional (3D) or a 2D image representing first to third layers of a tissue structure of the object-under-test 2. The tissue may be a skin tissue of a human or an animal body or a tissue structure of other objects.

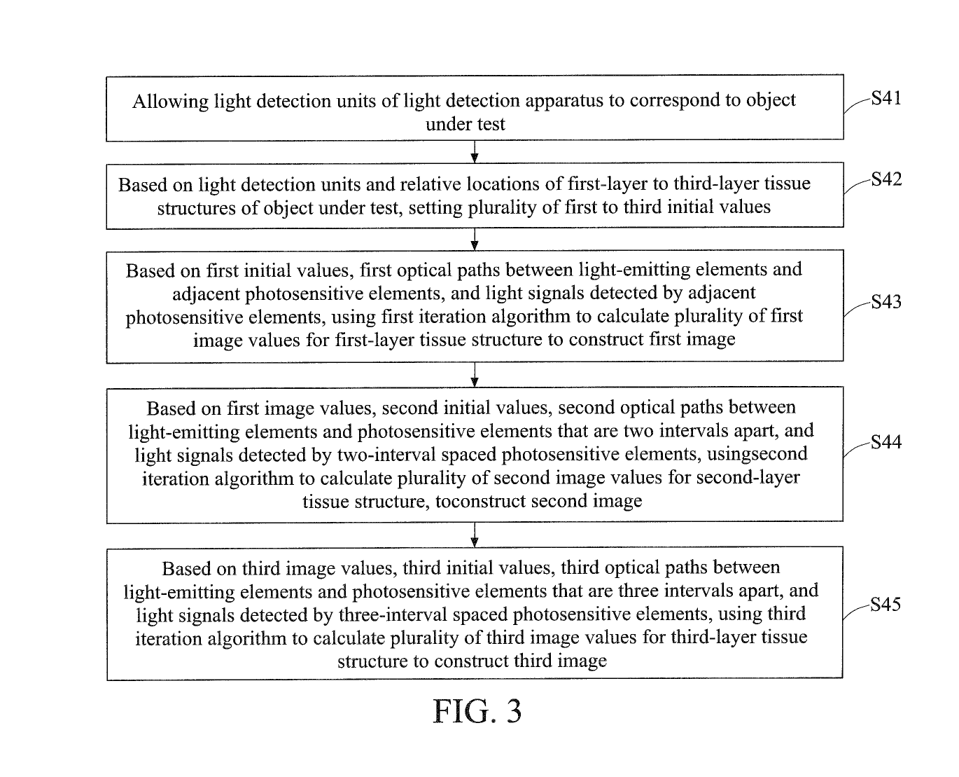

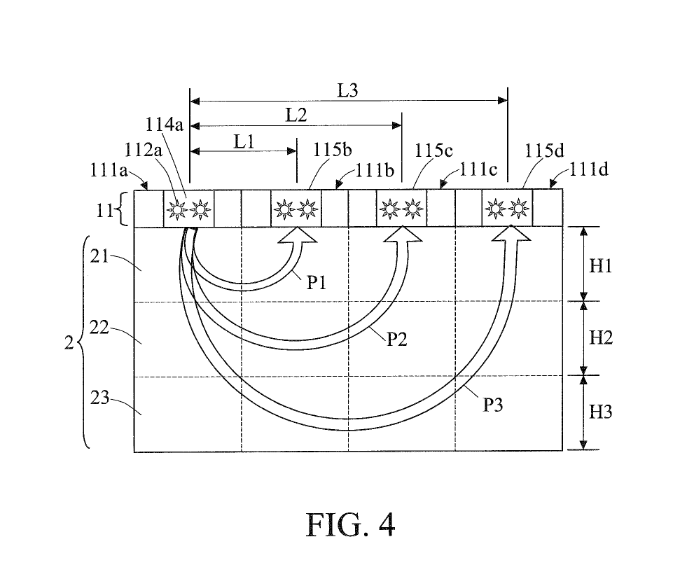

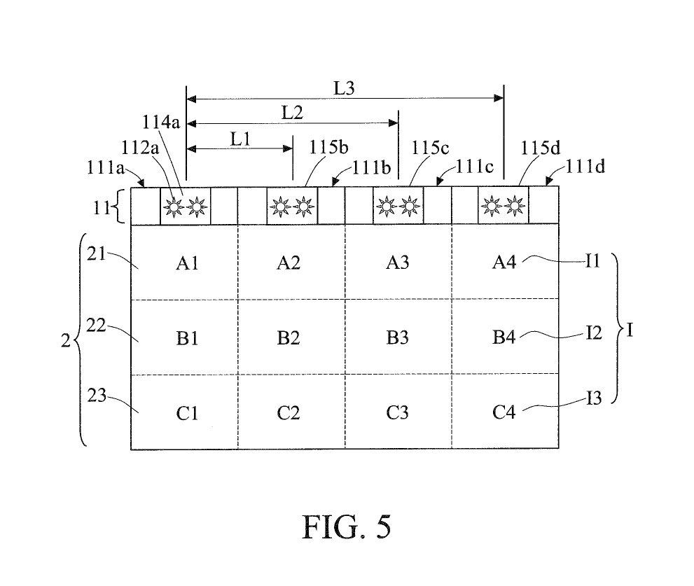

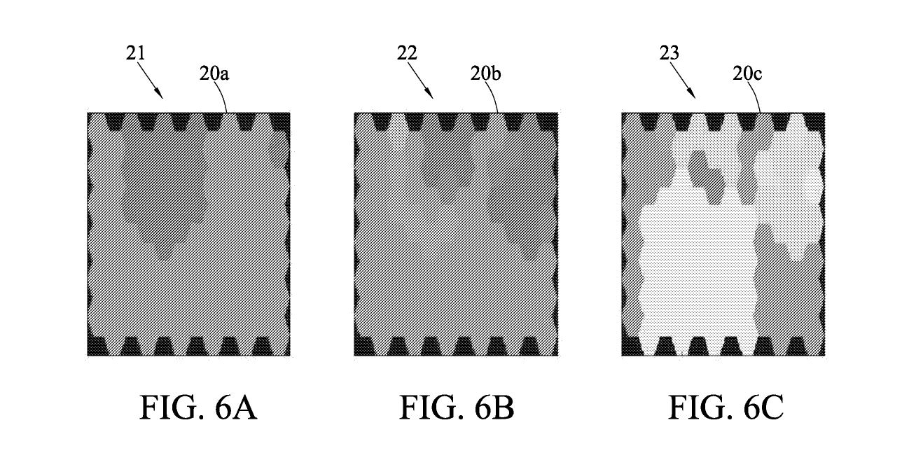

FIG. 3 is a flowchart illustrating an image reconstruction method using the light detection apparatus 1 shown in FIGS. 1 and 2 in accordance with the present invention. FIG. 4 is a schematic diagram depicting the detection module 11 of FIG. 2 corresponding to the object-under-test 2 and a first optical path P1 to a third optical path P3 in accordance with the present invention. FIG. 5 is a schematic diagram depicting the detection module 11 of FIG. 2 corresponding to the object-under-test 2 and a plurality of first initial values I1 to third initial values I3 in accordance with the present invention. FIGS. 6A to 6C are schematic diagrams depicting a first image 20a for a first-layer tissue structure 21, a second image 20b for a second-layer tissue structure 22, and a third image 20c for a third-layer tissue structure 23, respectively, of object-under-test 2 in accordance with the present invention.

As shown in FIGS. 3 to 6C, the image reconstruction method in accordance with the present invention includes the following steps. In an embodiment, four light detection units 111a to 111d (i.e., 111a, 111b, 111c and 111d), four light-emitting elements 114a to 114d, and four photosensitive elements 115a to 115d shown in FIG. 2 are used as an example, and the light-emitting element 114a produces a light source 112a, while three photosensitive elements 115b to 115d receive the corresponding light signals. However, the present invention is not so limited.

In step S41 of FIG. 3, a light detection apparatus 1 such as the one shown in FIGS. 1 and 2 is provided, and the light detection units 111 of the detection module 11 are made to correspond or come into contact with an object-under-test 2 such as the one shown in FIG. 4. Then, the method proceeds to step S42 of FIG. 3.

In step S42 of FIG. 3, a plurality of second initial values I2 (e.g., B1 to B4), and a plurality of third initial values I3 (e.g., C1 to C4) such as those shown in FIG. 5 are set to form an array I based on the relative locations of the light detection units 111, the first-layer tissue structure 21 to the third-layer tissue structure 23, a plurality of first initial values I1 (e.g., A1 to A4). The values of the first initial values I1 to the third initial values I3 may be the same or different. The number of the first initial values I1 to the third initial values I3 may be adjusted according to the number of light-emitting elements 114 or the photosensitive elements 115.

The first-layer tissue structure 21 is located at a first depth H1 of the object-under-test 2 as shown in FIG. 4. The first depth H1 may represent a first depth range (e.g., 0 to 0.667 cm) or a specific depth (e.g., 0.667 cm). The second-layer tissue structure 22 is located at a second depth H2 of the object-under-test 2. The second depth H2 may represent a second depth range (e.g., 0.667 to 1.334 cm) or a specific depth (e.g., 1.334 cm), and the second depth H2 is deeper than the first depth H1. The third-layer tissue structure 23 is located at a third depth H3 of the object-under-test 2. The third depth H3 may represent a third depth range (e.g., 1.334 to 2 cm) or a specific depth (e.g., 2 cm), and the third depth H3 is deeper than the second depth H2. However, the tissue structure of the object-under-test 2 may have four, five, six or more layers. Then, the method proceeds to step S43 of FIG. 3.

In step S43 of FIG. 3, using on Beer Lambert Law, and based on the first initial values I1 (e.g., A1 to A4), the first optical path P1 between the light-emitting elements 114 (e.g., 114a) and adjacent photosensitive elements 115 (e.g., 115b), and the light signals 113 detected by these adjacent photosensitive elements 115 (referring to FIG. 1), a first iteration algorithm (e.g., a non-linear iteration algorithm) is used to calculate a plurality of first image values for the first-layer tissue structure 21, and the first images values are repeated amended until the first image values are smaller than a first threshold such that the first image values are converged at the same time, and the (3D or 2D) first image 20a such as that shown in FIG. 6A is reconstructed based on these first image values. Then, the method proceeds to step S44 of FIG. 3.

In step S44 of FIG. 3, based on the first image values of the first-layer tissue structure 21, the second initial values of the second-layer tissue structure 22, the second optical path P2 between the light-emitting elements 114 (e.g., 114a) and photosensitive elements 115 spaced two intervals L2 apart (e.g., 115c), and the light signals 113 detected by these two-interval spaced photosensitive elements 115, a second iteration algorithm (e.g., a non-linear iteration algorithm) is used to calculate a plurality of second image values for the second-layer tissue structure 22, and the second images values are repeated amended until the second image values are smaller than a second threshold such that the second image values are converged at the same time, and the (3D or 2D) first image 20b such as that shown in FIG. 6B is reconstructed based on these second image values. Then, the method proceeds to step S45 of FIG. 3.

In step S45 of FIG. 3, based on the second image values of the second-layer tissue structure 22, the third initial values of the third-layer tissue structure 23, the third optical path P3 between the light-emitting elements 114 (e.g., 114a) and photosensitive elements 115 spaced three intervals L3 apart (e.g., 115d), and the light signals 113 detected by these three-interval spaced photosensitive elements 115, a third iteration algorithm (e.g., a non-linear iteration algorithm) is used to calculate a plurality of third image values for the third-layer tissue structure 22, and the third images values are repeated amended until the third image values are smaller than a third threshold such that the third image values are converged at the same time, and the (3D or 2D) third image 20c such as that shown in FIG. 6C is reconstructed based on these third image values.

From the above, it can be known that, in the light detection apparatus of the present invention, the light detection units of the detection module are constructed in such a way that they form a hexagonal or honeycomb array structure, so that the light source of each light-emitting element corresponds to photosensitive elements in six different directions simultaneously based on the characteristic of closely stacked hexagons. Therefore, the light detection apparatus is able to detect more light signals from the object-under-test, thus enabling fast reconstruction of the image of the object-under-test, and at the same time allowing the image of the object-under-test to have high resolution. Meanwhile, the light detection apparatus is portable and low cost, and is capable of Multiple-Input Multiple Output (MIMO) through the plurality of light-emitting elements and the plurality of photosensitive elements.

Furthermore, in the image reconstruction method using the light detection apparatus of the present invention, in addition to capable of detecting more light signals from the object-under-test, a first image of a first-layer tissue structure to a third image of a third-layer tissue structure of the object-under-test can be respectively constructed based on the first to the third iteration algorithms, thus facilitating the reconstruction of an image (e.g., a 3D image) of the object-under-test that is three layers deep.

In addition, the light detection apparatus of the present invention and the image reconstruction method using the same can be applied to diffuse optical tomography (DOT) systems, remote real-time monitoring care systems (such as home healthcare systems), relevant medical systems or other areas in order to provide the detections of breast cancer lesions or hemorrhagic stroke or the verification of brain functions, allowing users (such as physicians) to determine if the tissue structures of the object-under-test are normal or not based on these images and to quickly grasp a patient's condition or have real-time information concerning the situation of an individual being looked after.

The above embodiments are only used to illustrate the principles of the present invention, and should not be construed as to limit the present invention in any way. The above embodiments can be modified by those with ordinary skill in the art without departing from the scope of the present invention as defined in the following appended claims.

What is claimed is:

1. A light detection apparatus, comprising:
a detection module including a plurality of light detection units forming a hexagonal or honeycomb array structure, each of the light detection units including at least one light-emitting element and a photosensitive element; and
a control module connected with the detection module and including at least one selector and a multiplexer, wherein the selector selects at least one of the light-emitting elements of the light detection units to allow the selected light-emitting element to produce a light source and emit a plurality of photons to an object-under-test, and the multiplexer selects at least one of the photosensitive elements of the light detection units to allow the selected photosensitive element to detect light signals of the photons diffused to the object-under-test.

2. The light detection apparatus of claim 1, wherein each of the light detection units has a hexagonal grid or border, and each light-emitting elements of the light detection units is adjacent to at most six photosensitive elements.

3. The light detection apparatus of claim 1, wherein the light-emitting elements or the photosensitive elements in the same row of the light detection units are closely spaced at intervals of multiple increments.

4. The light detection apparatus of claim 1, wherein each of the light-emitting elements of the light detection units includes two light-emitting diodes that provide two light sources with two wavelengths, and the control module includes two selectors that control the two light sources of the light-emitting element of the light detection unit.

5. The light detection apparatus of claim 1, wherein the multiplexer is connected with the photosensitive elements of the light detection units, and receives light signals detected by the photosensitive elements.

6. The light detection apparatus of claim 5, further comprising a conversion module connected with the multiplexer and converting light signals from light intensity signals to voltage signals.

7. The light detection apparatus of claim 6, further comprising a processing module connected with the conversion module and constructing an image of a tissue structure of the object-under-test based on the voltage signals converted by the conversion module.

8. An image reconstruction method using the light detection apparatus of claim 1, comprising:
corresponding the light detection units of the light detection apparatus to the object-under-test;
setting a plurality of first initial values based on a relative location of the light detection units with respect to a first-layer tissue structure of the object-under-test at a first depth; and
using a first iteration algorithm to calculate a plurality of first image values for the first-layer tissue structure based on the first initial values, first optical paths between the light-emitting elements and adjacent photosensitive elements, and the light signals detected by the adjacent photosensitive elements, to amend the first images values repeatedly until the first image values are smaller than a first threshold, and constructing a first image based on the first image values.

9. The image reconstruction method of claim 8, further comprising:
setting a plurality of second initial values based on a relative location of the light detection units with respect to a second-layer tissue structure of the object-under-test at a second depth; and
using a second iteration algorithm to calculate a plurality of second image values for the second-layer tissue structure based on the first image values, the second initial values, second optical paths between the light-emitting elements and photosensitive elements that are spaced apart at two intervals, and the light signals detected by the two-interval spaced photosensitive elements, to amend the second images values repeatedly until the second image values are smaller than a second threshold, and constructing a second image based on the second image values.

10. The image reconstruction method of claim 9, further comprising:
setting a plurality of third initial values based on the light detection units and the relative location of a third-layer tissue structure at a third depth of the object-under-test; and
using a third iteration algorithm to calculate a plurality of third image values for the third-layer tissue structure based on the third image values, the third initial values, third optical paths between the light-emitting elements and photosensitive elements that are spaced apart at three intervals, and the light signals detected by the three-interval spaced photosensitive elements, to amend the third images values repeatedly until the third image values are smaller than a third threshold, and constructing a third image based on the third image values.
